(19) United States
(12) Patent Application Publication
(10) Pub. No.: US 2024/0386258 A1
(43) Pub. Date: Nov. 21, 2024

(54) NEURONAL CELL CULTURES AS COMPUTE SUBSTRATES

(71) Applicant: MICROSOFT TECHNOLOGY LICENSING, LLC, Redmond, WA (US)

(72) Inventors: Weiwei YANG, Seattle, WA (US); Benjamin Franklin CUTLER, Seattle, WA (US); Whitney HUDSON, Woodinville, WA (US); Christopher Miles WHITE, Redmond, WA (US)

(21) Appl. No.: 18/367,392

(22) Filed: Sep. 12, 2023

Related U.S. Application Data
(60) Provisional application No. 63/503,400, filed on May 19, 2023.
(60) Provisional application No. 63/503,406, filed on May 19, 2023.
(60) Provisional application No. 63/503,655, filed on May 22, 2023.

(51) Int. Cl. G06N 3/06; C12N 5/0793; G06N 3/092; G16B 50/30

(52) U.S. Cl. CPC G06N 3/061; C12N 5/0619; G06N 3/092; G16B 50/30; C12N 2513/00

(57) ABSTRACT

This disclosure provides techniques and systems for using a neuronal cell culture such as an organoid as a compute substrate. The cell culture is communicatively connected to an electronic computing device that provides input signals and receives output signals from the cell culture. The connection may be implemented, for example, with a multi-electrode array (MEA). The neuronal cell culture may function as a reservoir in reservoir computing. The neuronal cell culture when functioning as a reservoir provides a fixed, non-linear system that receives inputs and generates outputs. The neuronal cell culture may also function as a spiking neural network (SNN) where the neurons are nodes and the activation potentials are spikes. Neuronal cell cultures provide compute substrates that are energy efficient, cost-effective to manufacture, biodegradable, and adaptable.

### PRIORITY APPLICATIONS

This application claims the benefit of and priority to U.S. Provisional Application No. 63/503,400, filed May 19, 2023; U.S. Provisional Application No. 63/503,406, filed May 19, 2023; and U.S. Provisional Application No. 63/503,655, filed May 22, 2023, the entire contents of which are incorporated herein by reference.

### BACKGROUND

The demand for computing power is increasing. For example, sophisticated and resource intensive Large Language Models (LLMs) are now used by millions of people every day. Enormous amounts of computational resources are required to train and use modern artificial intelligence such as LLM's. However, the capacity of computing hardware is not keeping up with the increased demand. The monetary and energy costs required to create and operate ever more powerful systems to serve an ever-greater number of users could be a barrier to further development and broad adoption of advanced artificial intelligence. Additionally, the projected future demand for computing resources will have significant energy and environmental costs unless more efficient computing systems are developed. There are simply not enough resources to provide ubiquitous and constant access to resource-intensive computing such as artificial intelligence.

To enable continued innovation in artificial intelligence and to support the anticipated increase in demand for computing power, alternative computing platforms are necessary. This disclosure is made with respect to these and other considerations.

### SUMMARY

This disclosure provides systems and techniques that use neuronal cell cultures as compute substrates. Most current artificial intelligence systems use neural networks that are mathematical representations of interactions inspired by the behavior of neurons. This disclosure describes ways in which actual neurons can be cultured and used in conjunction with conventional electronic computing components to replace artificial neural networks (ANN) with a biological neural network (BNN). Thus, computer systems described in this disclosure will include a “neural network” implemented with actual cells rather than mathematical computations performed by a processor.

Biological computation is a billion times more energy efficient than silicon computation. The adoption of biological analogs of machine learning—using neurons to create a neural network within a computer—is an energy efficient way to implement artificial intelligence. A BNN may be able to perform equivalent calculations to an ANN with only a small percentage of the energy consumption. Thus, use of neuronal cell cultures to perform computing can make broader access to high-powered computing resources practical.

A neuronal cell culture is coupled to a conventional electronic computing device. In one implementation, neuronal cells are cultured on a multi-electrode array (MEA) that provides electrical stimulation to the cells and detects electrical activity of the cells. An input device and output device couples the biological components to the electronic. The MEA is one example of an input and output device but there are other types of devices which can provide inputs to the neuronal cell culture and detect the output of neurons firing. There are also many possible types of neuronal cell cultures that could be used. One type is the cortical organoid or “mini brain” which is a three-dimensional structure formed from a cluster of neurons.

The neuronal cell culture may function in place of many different types of ANNs. For example, the neuronal cell culture may be used as a spiking neural network (SNM), a recurrent neural network (RNN), or a reservoir for reservoir computing. Input signals (e.g., electrical signals generated by electrodes) are provided by the electronic computing device to the neuronal cell culture. The cells respond and generate output signals that are detected and conveyed back to the electronic computing device. For example, the neuronal cell culture may be used in reservoir computing for dimensionality expansion by mapping signals into a higher dimensional computational space or for dimensionality reduction by mapping signals into a lower-dimensional computational space. Input signals to the neuronal cell culture may encode information using multiple different types of encoding schemes such as spatial encoding or temporal encoding.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 illustrates a hybrid cellular-electronic computing system that includes a neuronal cell culture connected to an electronic computing device.

FIG. 2 illustrates an MEA with electrodes to provide input and detect output from a neuronal cell culture grown on the MEA.

FIG. 3 illustrates the use of a neuronal cell culture as a reservoir in reservoir computing.

FIG. 4 shows an illustrative method for using a neuronal cell culture to perform a compositional task.

FIG. 5 is a computer architecture diagram illustrating a computing device architecture for a computing device capable of implementing aspects of the techniques and technologies presented herein.

### DETAILED DESCRIPTION

Instead of creating approximations of biological neurons in an electronic-computing architecture, this disclosure describes the use of a neuronal cell culture as a compute substrate to process information directly with actual neurons. This compute platform uses a neuronal cell culture as a piece of hardware that functions in conjunction with conventional silicon electronics. Neurons are remarkable among cells in their ability to propagate signals rapidly over large distances. They do this by generating characteristic electrical pulses called action potentials: voltage spikes that can travel down axons. With each stimuli a neuron builds more cell membrane potential and when the potential reaches activation threshold the neuron fires. The rate of firing is determined by the rate of stimulation, a natural leak, activation threshold, and a refectory period. The specific connections between individual neurons and methods for processing information within a neuronal cell culture does not need to be elucidated to use a neuronal cell culture as a compute substrate. It is sufficient for the neuronal cell cultures to respond to input signals in a predictable way.

A neuronal cell culture is a BNN. A BNN is a complex network of interconnected neurons that work together to process and transmit information. The neurons communicate with each other through synapses, where electrical and chemical signals are transmitted from one neuron to another. The strength and pattern of these connections can change over time, allowing the network to adapt to new information and experiences. Natural BNNs are responsible for a wide range of functions in the brain, including perception, movement, learning, and memory.

ANNs are inspired by BNNs, but there are some key differences between them. BNNs are made up of biological neurons, each with many connections to other neurons, while ANNs are simplified mathematical models inspired by BNN, usually much smaller in scale and with a fixed architecture. In BNNs, the strength of connections between neurons can dynamically change and can be modified by a variety of factors, including learning and experience, while in many types of ANNs the connections between neurons are fixed and determined by a set of weights learned during training. The flexibility and adaptability of BNNs provides multiple advantages.

The ability of BNNs to dynamically change makes it possible for networks formed from a neuronal cell culture to grow and adapt in a way that ANNs cannot. For example, a neuronal cell culture connected to a large number of sensors may detect high-level activity in different areas of the neuronal cell culture. If the activity shifts to another region, that may be interpreted as a coded difference. Adaptation by the cells may be used to detect changes in data distributions in a natural way. However, detection of these types of shifts in activity with a conventional ANN is a costly problem in machine learning. If the neuronal cell culture changes or adapts, sensors connected to the cell culture detect the change and other parts of the computing system may respond by changing the mapping of output signals.

Because BNNs are made from living tissue, they have the potential to grow and adapt over time, leading to increased computing ability and capacity. This makes them particularly useful for complex computational tasks. The neurons can grow and adapt unlike a piece of fixed electronic hardware. The neurons can naturally grow into the task thereafter performed in an adaptive way. Networks of neurons are able to perform self-healing by regenerating damaged cells and rebuilding pathways between the cells. For example, if a neuron dies, neural pathways will be re-routed so that the neuronal cell culture as a whole still maintains the same functional mapping between inputs and outputs. The task is completed with different neurons and different pathways. This ensures the integrity of operation. These features make BNNs well-suited as a component of a compute substrate.

BNNs formed from neuronal cell cultures are adaptable due to the inherent flexibility of neural networks. For example, when encountering out-of-distribution data that has changed characteristics the neuronal cell cultures can adapt appropriately. With an ANN, out-of-distribution data may result in bad predictions because the weights of the network are adjusted to data that has a different distribution. However, with a BNN, the patterns of signaling in the neuronal cell culture will naturally change enabling this type of compute substrate to adapt to changing data distributions.

Additionally, unlike electronic components used for ANNs, BNNs are biodegradable. Their use will have less impact on the environment, and thus, they may be more sustainable. There are recycling and composting options available for biological material that are not available for conventional electronic hardware.

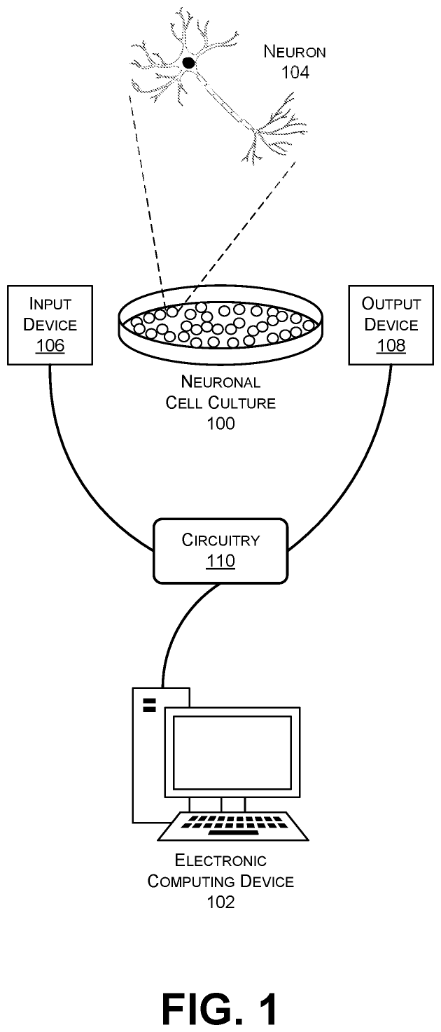

FIG. 1 illustrates a neuronal cell culture 100 coupled to an electronic computing device 102 forming a hybrid biological-electronic computing system. There are many techniques known to those of ordinary skill in the art for culturing neurons 104 to grow outside of a living organism. The neurons 104 may be grown from embryonic stem cells or induced pluripotent stem cells (iPSCs). The embryotic stem cells are guided using techniques known in the art (typically through chemical concentrations) to activate and/or inhibit genes that result in directed development into differentiated stem cells. Induced pluripotent stem cells (iPSCs) are a type of pluripotent stem cell that can be generated directly from a somatic cell. They are derived from skin or blood cells that have been reprogrammed back into an embryonic-like pluripotent state that enables the development of an unlimited source of any type of human cell needed. The neurons 104 may be human or from a non-human animal such as a primate or rodent. For example, the neurons 104 may be human induced pluripotent stem cells (hiPSCs). As a further example, the neurons 104 may be harvested from embryonic rodent brains. A neuronal cell culture 100 will typically contain a large number of individual neurons 104. In some implementations, the neurons 104 all come from the same source and are genetically identical. However, it is also possible to mix neurons 104 from different sources to create a neuronal cell culture 100 from two or more different types of neurons 104. Examples of suitable neurons 104 and techniques for creating neuronal cell cultures 100 are described in Cleber A. Trujillo et al., Complex Oscillatory Waves Emerging from Cortical Organoids Model Early Human Brain Network Development, 25 Stem Cell Reports 558 (2019).

The neuronal cell culture 100 may be a two-dimensional (2D) cell culture or a three-dimensional (3D) cell culture. In 2D cell cultures, cells are grown in a single layer on top of a flat surface, whereas in 3D cell cultures, the neurons 104 are grown in a 3D space. The formation of a cell monolayer in a 2D cell culture is faster and has lower reagent costs than in a 3D cell culture. However, 3D cell cultures mimic in vivo environments more closely and are typically longer lived than 2D cell cultures. A 3D cell culture is grown on a scaffolding that provides a structure for the cells to grow. The scaffolding may be created with sensors that become embedded in the cell culture as the subculture grows around the structures. An organoid is a specific type of 3D cell culture. Cancer tumorspheres are another example of a 3D cell culture.

An organoid is a miniaturized and simplified version of an organ produced in vitro in 3D that mimics the key functional, structural, and biological complexity of that organ. Organoids are derived from one or a few cells from a tissue, embryonic stem cells, or induced pluripotent stem cells, which can self-organize in three-dimensional culture owing to their intrinsic properties and/or extrinsic cues provided by the culture environment. Organoids are 3D cell cultures that contain organ-specific cell types which exhibit spatial organization and replicate some functions of the organ. Cortical organoids may exhibit cortical folds as well as vascularization.

Cortical organoids or brain organoids are one example of an organoid. Cortical organoids are derived from embryonic stem cells (ESCs) or iPSCs. Cortical organoids are created by culturing stem cells in a 3D rotational bioreactor and develop over months with cell types and cytoarchitectures that resemble an embryonic brain. The growth of a cortical organoid tends to reproduce the developmental path of the brain of a developing embryo. A cortical organoid grown from human cells is referred to as a human cortical organoid (HCO). Techniques for growing HCOs are known to those of ordinary skill in the art and described in Trujillo et al.

The neuronal cell culture 100 may be used as a compute substrate as described herein without prior training. Development of a neuronal cell culture 100 before use is different from training because the neuronal cell culture 100 is not directed to learning a new behavior while developing. However, it is also possible that the neuronal cell culture 100 will be trained prior to use as a compute substrate. The training may be performed, for example, by reinforcement learning in which the neuronal cell culture 100 is exposed to positive and/or negative feedback in response to goal-directed behaviors. Additionally, specific portions of the neuronal cell culture 100 may be trained or conditioned to differentiate into a particular type of neuron such as a sensory neuron or a motor neuron.

Once a neuronal cell culture 100 is prepared for use it can be connected to the electronic computing device 102 through an input device 106 and an output device 108. The input device 106 is configured to provide a stimulus or input that is detected by and causes a response by at least some of the neurons 104 in the neuronal cell culture 100. There are many different ways to provide input to the neuronal cell culture 100 including, but not limited to, electricity, light, chemical, sound, motion, and heat. Electrical stimulation may be provided to the neurons 104 with an electrode such as, but not limited to, an electrode that is part of an MEA. Excitation caused by activation of an electrode may increase cell membrane potential to a point that causes the neuron 104 to discharge. Light may be provided by LEDs, a projector, a digital micromirror, or other light source. In some implementations, the light may be provided as strobing light, for example a light that strobes at a frequency of 10 Hz.

Chemical simulation may be provided by applying the chemical directly to neurons 104 in the neuronal cell culture 100. Chemical stimulation also be applied to vascularized portions of the neuronal cell culture 100 that can carry the chemical signal. The chemical may, for example, be applied in a solution, powder, or solid. The chemical may be applied by any conventional means for applying chemical to cells in cell culture such as manual application with a dropper or pipette or automated application link that using laboratory robotics for microfluidics. Sound may be provided by one or more speakers or noisemakers. Motion may be provided by a vibrating tray, an agitator, or even by mechanically applying force (e.g., “poking”) neurons 104 in the neuronal cell culture 100. Heat may be applied by any conventional means for heating cells in cell culture such as use of resistors to create localized warming, application of warm fluids, microwaves, and the like.

The input device 106 may provide localized simulation to only a portion of the neurons 104 in the neuronal cell culture 100. Thus, input signals from the input device 106 can be provided to one or more specific regions of the neuronal cell culture 100. It is also possible to stimulate the neuronal cell culture 100 on a subcellular level.

The output device 108 is configured to detect activity of at least some of the neurons 104 in the neuronal cell culture 100. Depending on the specific type and configuration of the output device 108, output signals may be detected at one or more locations on the neuronal cell culture 100. There are many different ways to detect action potentials of a neuron 104.

In one implementation, the output device 108 is one or more electrodes. The electrodes may be bidirectional electrodes capable of both stimulating the neurons and detecting activation of the neurons. For example, the output device 108 may be implemented as an MEA that detects electrical signals generated when there is an action potential in a neuron 104. In other implementations, thermal sensors may be used to detect changes in the temperature of the neurons 104 and optical sensors may be used to measure neural activity based on surface plasmon resonance. See Mitra Abedini et al., Recording Neural Activity Based on Surface Plasmon Resonance by Optical Fibers—A Computational Analysis, 12 Front. Comput. Neurosci., 16 Oct. 2018). The input device 106 and output device 108 may be different devices and use different techniques for interfacing with the neuronal cell culture 100. For example, the input device 106 may apply a chemical to the neuronal cell culture 100 to provide input signals while the output device 108 detects temperature changes.

In some implementations, a single device functions as both the input device 106 and the output device 108. For example, an MEA may serve as both an input device 106 and an output device 108. A single bidirectional electrode may also serve as both an input device 106 and output device 108. When neurons 104 in the neuronal cell culture 100 generate an action potential, they produce electrical signals that can be detected by electrodes in the MEA. Examples of suitable MEAs, and techniques for growing a neuronal cell culture 100 on an MEA are known to those of ordinary skill in the art and described in Trujillo et al., Francesca Puppo et al., Super-Selective Reconstruction of Causal and Direct Connectivity With Application to in vitro iPSC Neuronal Networks, 15 Frontiers in Neuroscience (2021); and Brett J. Kagan et al., In vitro neurons learn and exhibit sentience when embodied in a simulated game-world, 23 Neuron 110, 3952-3969.e8 (2022).

Circuitry 110 may be used to couple the input device 106 and output device 108 with the electronic computing device 102. The specific type and function of the circuitry 110 will depend upon the type of input device 106 and output device 108 used. If the input device 106 and output device 108 are implemented as an MEA, the circuitry 110 provides an electrical and communicative connection between the electrodes of the MEA and the electronic computing device 102. The circuitry 110 may be any type of circuitry conventionally used to control an MEA or alternative type of sensor and stimulator. Control systems for MEAs are known to those of ordinary skill in the art and described in Trujillo et al., Puppo et al., Kagan et al., and Tianyi Chen et al., Discovering a Change Point and Piecewise Linear Structure in a Time Series of Organoid Networks via the Iso-Mirror, Applied Network Science 8:45 (2023). Control systems, software, MEA's, and accompanying circuitry are available from commercial sources including Axion BioSystems (Atlanta GA, USA).

The electronic computing device 102 can be any type of conventional computing device such as a desktop computer, laptop computer, tablet computer, smart phone, or the like. The electronic computing device 102 may also be physically located at a distance from the neuronal cell culture 100. For example, the electronic computing device 102 may be a network-accessible or cloud-based computing device with physical components spread across multiple different locations. In an implementation, the circuitry 110 is connected to a network and signals from the circuitry 110 are conveyed through the network to the electronic computing device 102.

The electronic computing device 102 includes software components that drive the circuitry 110 as well as interpret signals received from the circuitry 110. The software converts input data into instructions that cause the input device 106 to generate a specific input signal to the neuronal cell culture 100. Output signals detected by the output device 108 and provided to the electronic computing device 102 are interpreted by the software and converted into output data. Thus, the software is used to encode information in a way that can be provided to the neuronal cell culture 100 and to decode patterns of action potentials detected by the output device 108.

With this configuration, the neuronal cell culture 100 can be integrated with the electronic computing device 102 creating a computing system that includes both biological and electronic compute substrates. As hardware component in a computing system, the neuronal cell culture 100 may be thought of as a black box that receives input data and generates output data. The behavior and connections of the neurons 104 in the neuronal cell culture 100 map an input to an output. If the responses are predictable or deterministic, they can be mapped back to the stimulus. This represents a meaningful change that enables the use of the neuronal cell culture 100 for performing computation. Thus, digital information from the electronic computing device 102 is converted to appropriate signals, transformed by the neuronal cell culture 100, and returned from the output device 108 as different digital information. This output can then be processed further by the conventional electronic computing device 102.

In some implementations, multiple neuronal cell cultures 100 can be chained together so that the output from one provides input to the next. Thus, signals will pass from one or more input devices 106 to a first neuronal cell culture then to a second and possibly additional neuronal cell cultures before being detected by one or more output devices 108. When multiple neuronal cell cultures 100 are used together, each may include the same design type of cells or they may be different. For example, a first neuronal cell culture may use human cells while a second uses mouse cells and a third may use a combination of cells from different origins.

In addition to the possibility of using different types of input devices 106, there are multiple ways that stimulus to the neuronal cell culture 100 can be controlled in order to encode input signals. Stimulus to the neuronal cell culture 100 can be varied using any one or more of location, intensity, and timing to encode input signals. Spatial or placement encoding encodes information with the location of the stimulus. Use of the input device 106 to stimulate different portions of the neuronal cell culture 100 can encode different types of information. For example, if the input device 106 is an MEA, activating only certain electrodes controls the location of stimulation. If intensity is used for encoding, the strength of the stimulus provided by the input device 106 is used to encode information. For example, if the input device 106 is an electrode the voltage of an electrical shock can be varied to control the strength of stimulus.

In temporal encoding, information is represented by the relative timing of stimulus. The frequency or periodicity of stimulus may be used to encode information. For example, if the input device 106 uses light, the timing in which light is applied or frequency at which the light flashes can be used to encode input signals. A light flashing at a first frequency would encode a different signal than a light flashing with a different frequency. Neurons 104 in the neuronal cell culture 100 may also use temporal encoding to pass signals amongst themselves. Neurons 104 communicate through action potentials at specific times, which adds an intrinsic temporal aspect to their information-processing capabilities. Temporal encoding allows for learning through backpropagation with exact derivatives and achieves accuracies on par with ANNs. When precise spike timing or high-frequency firing-rate fluctuations are found to carry information, the neural code is often identified as a temporal code.

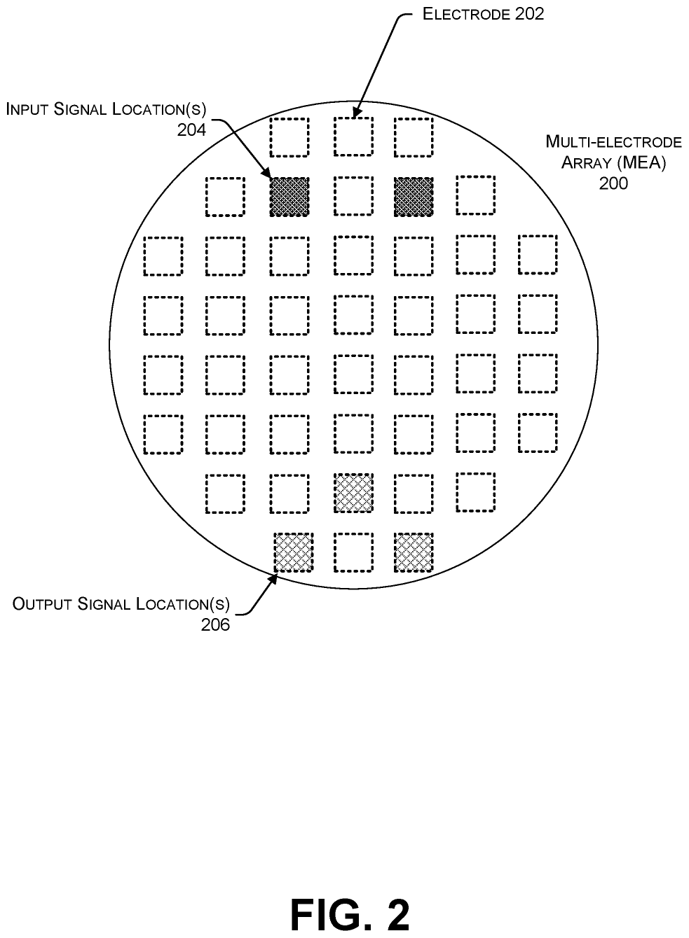

FIG. 2 illustrates an MEA 200 that functions as a substrate on which a neuronal cell culture is grown. The neuronal cell culture is not shown in FIG. 2. When an MEA 200 is used as a substrate, the neurons are grown in contact with one of the electrodes that make up the MEA 200. An MEA 200, such as a high-density MEA, may have a large number of electrodes 202 in contact with the neurons of the neuronal cell culture.

The MEA 200 may have only a single well or it may be an MEA plate having multiple wells such as 8, 16, 24, 32, 48, or another number of wells. In one implementation, each well in the plate contains 16, 24, 32, 48, or 64 low-impedance (0.04 MU) platinum microelectrodes with 30 mm of diameter spaced by 200 mm. One source of MEA plates is Axion Biosystems of Atlanta, Georgia, USA. A single well may contain one, two, or three, or more organoids. In one implementation, the MEA may be a MaxOne MEA from Maxwell Biosystems, Switzerland that has 26,000 platinum electrodes arranged over an 8 mm² area. The MaxOne system is based on complementary meta-oxide-semiconductor (CMOS) technology and allows recording from up to 1024 channels.

One or more of the electrodes 202 in the MEA 200 are used as input signal locations 204 to provide stimulus to the neurons in the form of electrical shocks. One or more other electrodes 202 are used as output signal locations 206 to detect action potentials. Both the number and location of the input signal locations 204 and the output signal locations 206 may be varied. Changes to input signal locations 204 and output signal locations 206 can be made while a neuronal cell culture is functioning as a compute substrate.

In some implementations, input signals are provided at input signal locations 204 to a first region of the neuronal cell culture and a deterministic response is detected as output signals at output signal locations 206 in contact with a different region of the neuronal cell culture. As mentioned above, multiple different aspects of input signals can be varied to encode information. The choice of input signal locations 204 is one way information can be encoded. Intensity, timing, and frequency of the electrical stimuli generated by electrodes 202 may also be varied to encode information. The input signal locations 204 are not fixed and may be intentionally changed. For example, providing impulses into a first region of the neuronal cell culture may be used to encode something different than providing impulses to a second region of the neuronal cell culture.

The output signal locations 206 can be detected in a different portion of the neuronal cell culture than the input signal locations 204. A change in inputs typically results in different outputs. The nature of the difference is a result of the “computation” performed by the neuronal cell culture. However, the specific neuronal pathways and signaling that occur between the locations of inputs and outputs may be unknown. The neuronal cell culture can provide functions similar to that of an ANN. However, features such as the number of layers, the level of interconnection, and the weights of connection do not need to be designed by hand. Rather, the type of cells used for the neuronal cell culture, the techniques for culturing the cells, the structure of the cells, the level of maturity of the cell culture, the techniques for providing inputs, and the locations of the output signal locations 206 are modified by a designer of the system. Neuronal cell cultures provide deterministic output because they can produce macro behavior that results in the same output signals without randomness or variability every time the same input is provided.

Use of an MEA 200 (or other types of input devices and output devices with similar addressability) allows for flexibility in managing the interface between the neuronal cell culture and an electronic computing device. For example, if there is damage or a change to the neuronal cell culture, the input signal locations 204 and/or the output signal locations 206 may be changed to maintain consistent deterministic behavior of the neuronal cell culture. If the output signal locations 206 are fixed but there is some change to the neuronal cell culture, a reinforcement mechanism may be used to encourage the neuronal cell culture to adapt in a way that it continues to provide the mapped output to the same output signal locations 206.

Moreover, unlike conventional hardware chips with a set number of pins, a neuronal cell culture grown on MEA 200 may have an initially unknown number of output signal locations 206. For example, all or substantially all of the electrodes 202 in the MEA 200 may initially be used to detect voltage changes. However, after observing the behavior of the neuronal cell culture, specific electrodes 202 that detect meaningful action potentials in response to inputs can be identified. It is these electrodes that are then used as the output signal locations 206. Thus, locations on the neuronal cell culture detecting outputs can be flexibly identified by using multiple electrodes 202 in an MEA 200 rather than a more limited fixed set of sensors. It is also possible that the output signal locations 206 may be task dependent meaning that the output is detected in different portions of a neuronal cell culture depending on the type of inputs provided to the neuronal cell culture.

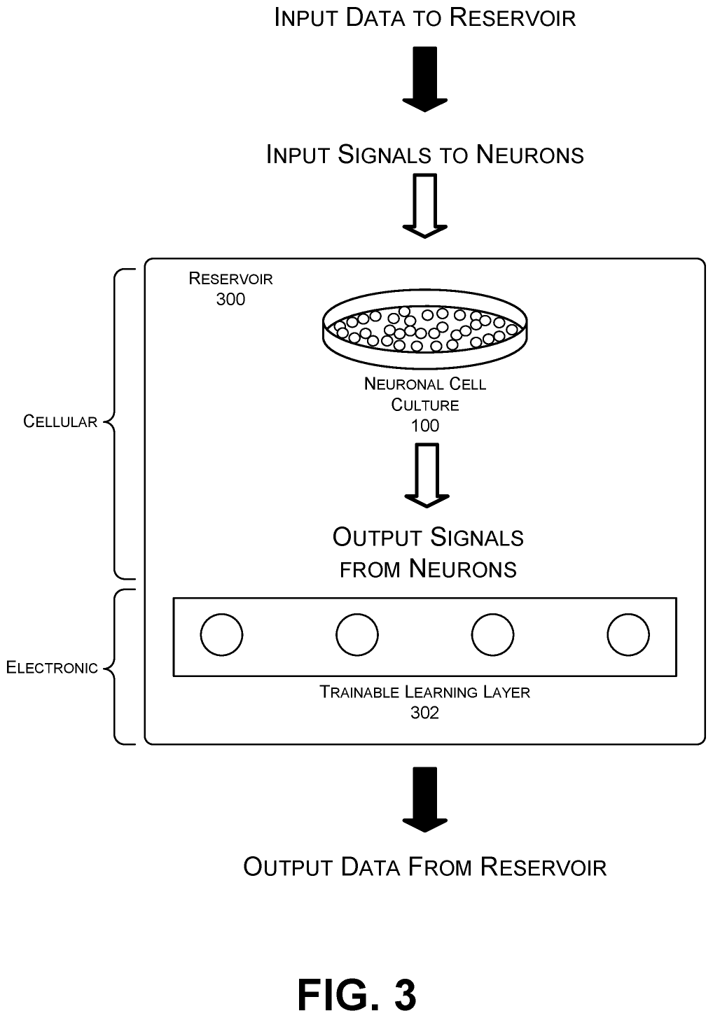

FIG. 3 illustrates the use of a neuronal cell culture 100 as a reservoir 300 in reservoir computing. Conventional reservoir computing is a machine learning technique that processes information generated by dynamical systems using observed time-series data. Reservoir computing is a computation framework used for information processing and supervised learning. In a typical electronic implementation, reservoir computing uses recurrent neural networks (RNN) to map input data into higher dimensional computational spaces through the dynamics of a fixed, non-linear system called a reservoir 300. The reservoir 300 is typically a random or structured dynamical system that is driven by the input signal and has a large number of internal degrees of freedom. The reservoir 300 acts as a “black box” that transforms the input data into a more complex representation that captures the temporal dynamics of the signal. The output data is then fed into a simple readout mechanism that is trained to map the reservoir state to the desired output.

Reservoir computing requires very small training data sets, uses linear optimization, and thus requires minimal computing resources. It has been used in various applications such as speech recognition, image classification, and time-series prediction. reservoir computing has attracted significant attention in various research fields because it is capable of fast learning that results in reduced computational/training costs compared to other RNNs. One advantage of reservoir computing is that only the output weights (readout weights) are trained using a simple learning rule, realizing a fast-learning process, and enabling a reduction in computational cost.

Suitable reservoirs are not limited to software implementations of RNNs. Mechanisms like 2-D quantum dot lattices, nuclear spins in a molecular solid and echo-state networks have all successfully been used as a reservoir in reservoir computing. Previous work has also demonstrated the effectiveness of optoelectronic reservoirs based on photonic materials to successfully solve classical machine learning problems. Kazutaka Kanno and Atsushi Uchida, “Photonic reinforcement learning based on optoelectronic reservoir computing,” 12 Scientific Reports 3720 (2022). Photonic reservoir computing is a type of computing that uses light to perform general signal processing tasks. The optoelectronic reservoirs were not biologically based but included neuron-like adaptive elements. Kanno and Uchida describe a photonic on-line implementation of reinforcement learning using optoelectronic delay-based reservoir computing. Although this does not use neuronal cell cultures, it is an example of a non-software component with neuron-like elements functioning as a reservoir in a machine learning application.

Highly coupled, nonlinear electronic networks have also been used as alternatives to RNNs for reservoir computing. These electronic networks exhibit emergent criticality similar in nature to previously reported electrical activity of biological brains and neuron assemblies. The structure of the highly coupled, nonlinear electronic networks is similar to a Turing B-Type unorganized machine. Stieg, A. Z., Avizienis, A. V., Sillin, H. O., Martin-Olmos, C., Aono, M. and Gimzewski, J. K. (2012), Emergent Criticality in Complex Turing B-Type Atomic Switch Networks. Adv. Mater., 24:286-293. This is a further example of an alternative compute substrate besides a conventional ANNs functioning as a reservoir 300 in reservoir computing.

Neuronal cell cultures 100 are well suited to function as a natural reservoir to be combined with conventional computing techniques because they provide a very large number of interconnected nodes—the neural cells. As a neuronal cell culture 100 reacts to a stimulus and by utilizing the complex electro-physiological activities already present in these cells, input signals can be mapped into higher or lower dimensional spaces. In order to serve as a compute substrate, such as a reservoir 300 for reservoir computing, neural cell cultures do not necessarily need to be organized into specific structures. Even cells in a disorganized form can still perform cellular communication. This approach is attractive because it does not require understanding the mechanism of learning in order to use the behavior of the neuronal cell culture 100 to perform a computation.

Input data is provided to the reservoir 300. This input data may be the same as it would be in a conventional electronic computing device that uses a reservoir 300 implemented entirely with an ANN. The input data is converted into signals to neurons in the neuronal cell culture 100. This may include use of circuitry and an input device to encode the input data in a form that can be delivered to the neurons.

In one implementation, the reservoir 300 may consist only of a neuronal cell culture 100. Output signals from neurons in the neuronal cell culture 100 are then converted into the output data from the reservoir 300. This conversion may be performed as described above using an output device to detect activation potentials which are then passed through circuitry to an electronic computing device that decodes the output signals into output data. This output data may be in the same form as output data from an ANN used in conventional reservoir computing.

In the implementation illustrated in FIG. 3, the reservoir 300 includes the neuronal cell culture 100 as well as a trainable learning layer 302 implemented in software or a specially constructed circuit. With this configuration, the internal synaptic behavior serves as a high-dimensional substrate for information processing and the output signals from the neurons are provided to an electronic computing device that processes those outputs through the trainable learning layer 302. The trainable learning layer 302 may be implemented as a single layer of an ANN. In this implementation, the output from the trainable learning layer 302 becomes the final output data from the reservoir 300. In some implementations, the trainable learning layer 302 may be implemented biologically with a separate neuronal cell culture.

The neuronal cell culture 100 may be used to change the dimensionality of an input. For example, the neuronal cell culture 100 may perform dimensionality reduction or dimensionality expansion. The neuronal cell culture 100 can fill the role of a basis function. Basis functions (called derived features in machine learning) are building blocks for creating more complex functions. In other words, they are a set of k standard functions, combined to estimate another function-one which is difficult or impossible to model exactly. This can be used to shrink the search space so that it is manageable in a small manifold. This can improve understanding of the input data when there is a complex problem that will benefit from a reduction in complexity such as a biological bloom filter. This is dimensionality reduction.

The neuronal cell culture 100 may also be used for performing dimensionality expansion. Dimensionality expansion may be needed if the reactions in the input space are too close together and more expressiveness is needed to fully understand the input data. The input data is passed through a complex system, the neuronal cell culture 100, that is deterministic and which expands the input space into a larger space in a nonlinear fashion. Dimensionality expansion allows for the separation of the parts of input data and the use of each part separately in the output data.

The neuronal cell culture 100 may also be used to provide a basis expansion function. A basis expansion function is a mathematical technique that transforms a set of input data into a higher-dimensional space to make it easier to model complex relationships between the input and output variables. Output devices in contact with multiple regions of the neuronal cell culture 100 may be used to read the response. This response can be analyzed to understand how the input signals map into a larger manifold space. The output signals from the neuronal cell culture 100 may then be used to provide bases inputs into a machine learning model or any other conventional software system.

Another way of leveraging the neuronal cell culture 100 is by using it directly as a spiking neural network (SNN). The flow of data and connections to the electronic computing device will be the same as described above. A conventional SNN is a type of ANN that models the behavior of biological neurons. Unlike traditional ANNs, which use continuous activation functions, spiking neural networks use discrete time-based events, or “spikes,” to communicate information between nodes rather than continuous signals. Thus, a biological substitute for a SNN can be built simply by using the natural spiking activity of the neurons in the neuronal cell culture 100.

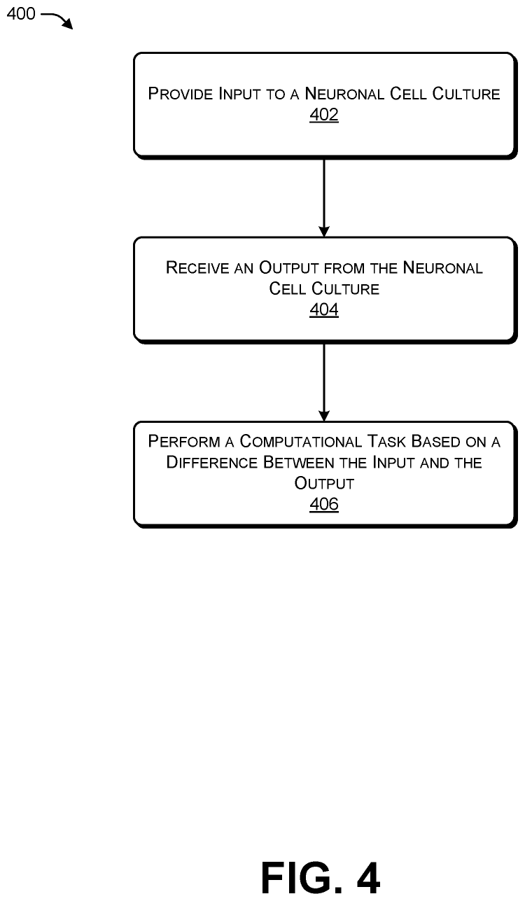

FIG. 4 shows a method 400 that uses a neuronal cell culture to perform a computational task. Method 400 may be performed using the systems and devices shown in FIGS. 1-3.

At operation 402, an input signal is provided to a neuronal cell culture. The input signal may be provided by circuitry coupled to an electronic computing device. For example, the input signal may be provided by a first electrode in contact with the neuronal cell culture at a first location. The first electrode may be an electrode in a MEA as shown in FIG. 2. The input signal may be provided by a single electrode or by multiple electrodes. The input signal encodes input data in a format that can be delivered and interpreted by the neuronal cell culture. The input signal may be encoded by weight encoding, spatial encoding, temporal encoding, frequency encoding, or another type of encoding.

At operation 404, an output signal is received from the neuronal cell culture. The output signal may be received from circuitry coupled to the electronic computing device. For example, the output signal may be received from a second electrode in contact with the neuronal cell culture at a second location. The second electrode may be an electrode in a MEA as shown in FIG. 2. The input signal may be detected by a single electrode or by multiple electrodes. In an implementation, the first electrode and the second electrode are electrodes that are both part of the same MEA. In one implementation, the output signal is decoded to output data.

At operation 406, a computational task is performed by the electronic computing device based on a difference between the input signal and the output signal. The computational task may be any type of computational task conventionally performed by computers. The computational task may be a subtask that is part of a larger task involving multiple subtasks. For example, in performing the computational task the neuronal cell culture may function as a reservoir for reservoir computing. The neuronal cell culture may also be used to perform the computational task by performing as a RNN or SNN.

Because neuronal cell cultures contain living cells, unlike electronic computing hardware, they can adapt and change to behave in new ways. Accordingly, interaction of the neuronal cell cultures with other parts of the system is dynamic and flexible.

The neuronal cell culture is able to adapt to variable amounts of input data provided through the input signals. This type of adaptation does not involve an explicit change in compute capacity nor external changes to the system housing the neuronal cell culture. However, the inherent flexibility and malleability of neurons makes it possible for increases in resolution in some regions of the neuronal cell culture. It is believed that changes in resolution may result from the neurons responding to an increase in inputs by dividing and responding in finer detail. This is believed to be possible because each neuron has thousands of synapses each can be used to convey information depending on its connectivity. There is an overabundance of expressiveness and is possible for new neuro-circles to be formed in adaptive response to new input. The adaption of the neuronal cell culture allows for an increase in data throughput without changing the neuronal cell culture or the electronic computing device. Thus, neuronal cell cultures can spontaneously change capabilities and increase capacity even with no other change to the system.

For example, at one point during the use of a neuronal cell culture as a compute substrate there may be five regions that exhibit different activation patterns. After being exposed to input signals for a period of time the number of distinctly active regions may increase to, for example, eight. This can happen without explicit direction from a user or from the electronic computing device. The changes in activation patterns are observed or detected and then the output device is updated. For example, the specific electrodes on an MEA that are used to detect activation potentials may change. Thus, even though computing capacity as such may not have changed, the entire system due to adaptation of the neuronal cell culture can process a greater amount of information. The new mapping pattern (e.g., eight regions instead of five) can correspond to new functionalities. However, as additional inputs are mapped into a neuronal cell culture there may come a point where the computational capacity of the neurons is saturated and processing bandwidth cannot increase further.

### Computing Devices and Systems

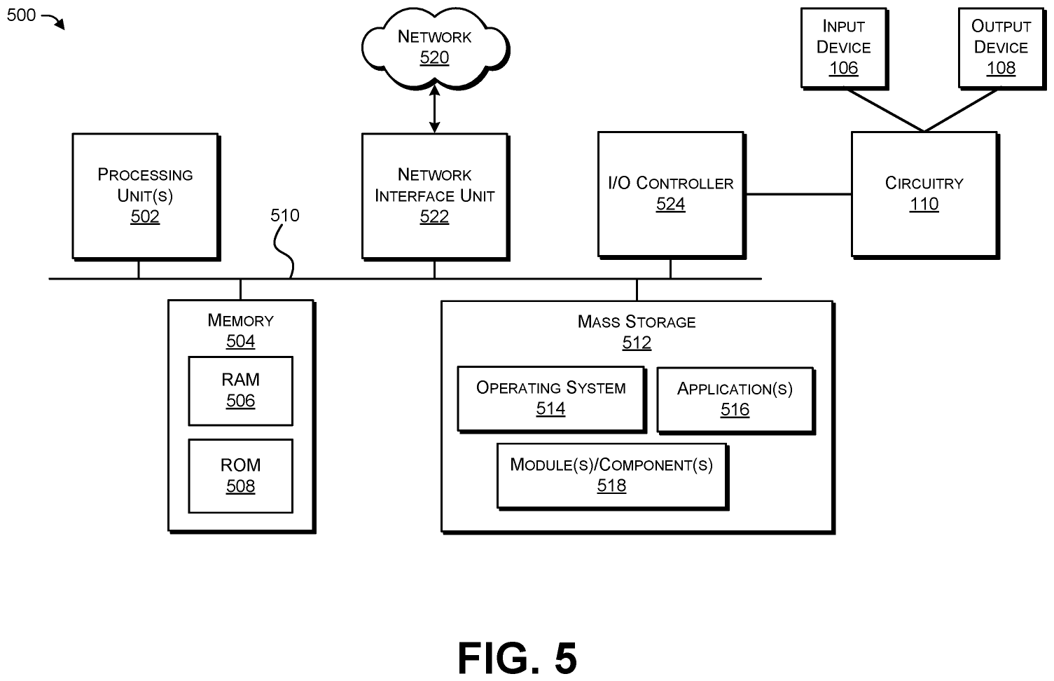

FIG. 5 shows details of an example computer architecture 500 for an electronic computing device such as the electronic computing device 102 introduced in FIG. 1. The computer architecture 500 may represent a computer or a server configured as part of a local or cloud-based platform, capable of executing computer instructions (e.g., a module or a component described herein). The computer architecture 500 may be a von Neumann architecture. The computer architecture 500 illustrated in FIG. 5 includes processing unit(s) 502, a memory 504, including a random-access memory 506 (“RAM”) and a read-only memory (“ROM”) 508, and a system bus 510 that couples the memory 504 to the processing unit(s) 502. The processing unit(s) 502 include one or more hardware processors and may also comprise or be part of a processing system. In various examples, the processing units 502 of the processing system are distributed. Stated another way, one processing unit 502 may be located in a first location (e.g., a rack within a datacenter) while another processing unit 502 of the processing system is located in a second location separate from the first location.

The processing unit(s) 502 can represent, for example, a CPU-type processing unit, a GPU-type processing unit, a field-programmable gate array (FPGA), another class of digital signal processor (DSP), or other hardware logic components that may, in some instances, be driven by a CPU. For example, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), and the like. The processing unit 502 may include both an arithmetic logic unit and processor registers.

A basic input/output system containing the basic routines that help to transfer information between elements within the computer architecture 500, such as during startup, is stored in the ROM 508. The computer architecture 500 further includes a mass storage 512 for storing an operating system 514, application(s) 516, modules/components 518, and other data described herein. The mass storage 512 may also include a control unit that includes an instruction register and a program counter. The application(s) 516 and the module(s)/component(s) 518 may implement communication of signals between the electronic computing device and a neuronal cell culture.

The mass storage 512 is connected to processing unit(s) 502 through a storage controller connected to the bus 510. The mass storage 512 provides non-volatile storage for the computer architecture 500. The mass storage 512 may be implemented as computer-readable media that can be any available computer-readable storage medium or communications medium accessible by the computer architecture 500.

Computer-readable media includes computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including RAM, static random-access memory (SRAM), dynamic random-access memory (DRAM), phase-change memory (PCM), ROM, erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network-attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer-readable storage medium does not include communication medium. That is, computer-readable storage media does not include communications media and thus excludes media consisting solely of a modulated data signal, a carrier wave, or a propagated signal, per se.

According to various configurations, the computer architecture 500 may operate in a networked environment using logical connections to remote computers through the network 520. The computer architecture 500 may connect to the network 520 through a network interface unit 522 connected to the bus 510. An input/output (I/O) controller 524 may also be connected to the bus 510 to control communication in input and output devices. One example of an I/O device is the circuitry 110 introduced in FIG. 1. Thus, the computer architecture 500 may be coupled through the I/O controller 524 to the input device 106 and the output device 108 that are in turn coupled to a neuronal cell culture. In one implementation, the input device 106 and the output device 108 are implemented as a MEA connected to the neuronal cell culture.

The circuitry 110, when configured for interfacing with an MEA, may include a bandpass filter and adaptive threshold spike detector. For example, the bandpass filter may be set to 10-25,000 Hz. In another implementation, the bandpass filter may be set to 0.1 Hz to 5 kHz. The adaptive threshold spike detector may be set to 5.5× standard deviations. In one implementation, the circuitry 110 may include a 2nd order high-pass Bessel filter with 100 Hz cut-off followed by a 1st order low-pass Bessel filter with 1 Hz cut-off. Raw data after being passed through the circuitry 110 can be acquired and processed with software such as the Maestro recording system and Axion Integrated Studio available from Axion Biosystems. Alternative software that may be used includes the AxIS Software Spontaneous Neural Configuration also from Axion Biosystems.

It should be appreciated that the software components described herein may, when loaded into the processing unit(s) 502 and executed, transform the processing unit(s) 502 and the overall computer architecture 500 from a general-purpose computing system into a special-purpose computing system customized to facilitate the functionality presented herein. The processing unit(s) 502 may be constructed from any number of transistors or other discrete circuit elements, which may individually or collectively assume any number of states. More specifically, the processing unit(s) 502 may operate as a finite-state machine, in response to executable instructions contained within the software modules disclosed herein. These computer-executable instructions may transform the processing unit(s) 502 by specifying how the processing unit(s) 502 transitions between states, thereby transforming the transistors or other discrete hardware elements constituting the processing unit(s) 502.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.
Clause 1. A computing system comprising: an electronic computing device (102); a neuronal cell culture (100); and an input device (106) and output device (106) configured to communicatively connect the electronic computing device and the neuronal cell culture, wherein the neuronal cell culture is configured to function as a compute substrate in conjunction with the electronic computing device.
Clause 2. The computing system of clause 1, wherein the electronic computing device comprises a processing unit (502), a memory (504), and a mass storage (512).
Clause 3. The computing system of clause 1 or 2, wherein the neuronal cell culture comprises differentiated embryonic stem cells or induced pluripotent stem cells.
Clause 4. The computing system of any of clauses 1 to 3, wherein the neuronal cell culture comprises a two-dimensional (2D) cell culture or a three-dimensional (3D) cell culture.
Clause 5. The computing system of clause 4, wherein the 3D cell culture is an organoid.
Clause 6. The computing system of any of clauses 1 to 5, wherein the input device and output device comprise electrodes (202) configured to provide electric signals to the neuronal cell culture as input signals and detect activation potentials of neurons in the neuronal cell culture as output signals.
Clause 7. The computing system of clause 6, wherein the electrodes are configured as a multi-electrode array (MEA) (200).
Clause 8. The computing system of clause 7, wherein the input signal is provided by first electrodes in the MEA contacting the neuronal cell culture at a first location and the output signal is detected by second electrodes in the MEA contacting the neuronal cell culture at a second location.
Clause 9. The computing system of clause 7, wherein the input signal and the output signal are provided and detected by one or more bidirectional electrodes at the same or different locations.
Clause 10. The computing system of any of clauses 1 to 9, wherein the computing system is configured such that the neuronal cell culture functions as a reservoir (300) in reservoir computing.
Clause 11. The computing system of clause 10, wherein the electronic computing device implements a trainable learning layer (302) that receives an output signal from the neuronal cell culture.
Clause 12. The computing system of clause 11, wherein the neuronal cell culture and the trainable learning layer together are configured to function as a recurrent neural network (RNN).
Clause 13. The computing system of any of clauses 10 to 12, wherein the reservoir is configured to map input signals received from the input device into a different computational space.
Clause 14. The computing system of any of clauses 1 to 8, wherein the computing system is configured such that the neuronal cell culture functions as a spiking neural network (SNN).
Clause 15. The computing system of clause 14, wherein activation potentials of neurons in the neuronal cell culture provides signals for the SNN.
Clause 16. A method of using a neuronal cell culture as a compute substrate comprising: providing, by circuitry (110) coupled to an electronic computing device (102), an input signal to the neuronal cell culture; receiving, by the circuitry coupled to the electronic computing device, an output signal from the neuronal cell culture; and performing a computational task by the electronic computing device based on a difference between the input signal to the neuronal cell culture and the output signal from the neuronal cell culture.
Clause 17. The method of clause 16, wherein the input signal is provided by a first electrode in contact with the neuronal cell culture at a first location and the output signal is received by a second electrode in contact with the neuronal cell culture at a second location or the input signal and output signal are provided and received by one or more bidirectional electrodes.
Clause 18. The method of clause 16 or 17, wherein information in the input signal is encoded by spatial encoding.
Clause 19. The method of clause 16 or 17, wherein information in the input signal is encoded by temporal encoding.
Clause 20. The method of any of clauses 16 to 19, wherein in performing the computational task the neuronal cell culture functions as a RNN or SNN.

### CONCLUSION

While certain example embodiments have been described, including the best mode known to the inventors for carrying out the invention, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein. Skilled artisans will know how to employ such variations as appropriate, and the embodiments disclosed herein may be practiced otherwise than specifically described. The accompanying claims and their equivalents are intended to cover such forms or modifications as would fall within the scope and spirit of certain of the inventions disclosed herein.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element (e.g., two different sensors).

In closing, although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

Furthermore, references have been made to publications, patents and/or patent applications throughout this specification. Each of the cited references is individually incorporated herein by reference for its particular cited teachings as well as for all that it discloses.

1. A computing system comprising:
an electronic computing device;
a neuronal cell culture; and
an input device and output device configured to communicatively connect the electronic computing device and the neuronal cell culture, wherein the neuronal cell culture is configured to function as a compute substrate in conjunction with the electronic computing device.

2. The computing system of claim 1, wherein the electronic computing device comprises a processing unit, a memory, and a mass storage.

3. The computing system of claim 1, wherein the neuronal cell culture comprises differentiated embryonic stem cells or induced pluripotent stem cells.

4. The computing system of claim 1, wherein the neuronal cell culture comprises a two-dimensional cell culture or a three-dimensional cell culture.

5. The computing system of claim 4, wherein the 3D cell culture is an organoid.

6. The computing system of claim 1, wherein the input device and output device comprise electrodes configured to provide electric signals to the neuronal cell culture as input signals and detect activation potentials of neurons in the neuronal cell culture as output signals.

7. The computing system of claim 6, wherein the electrodes are configured as a multi-electrode array (MEA).

8. The computing system of claim 7, wherein the input signal is provided by first electrodes in the MEA contacting the neuronal cell culture at a first location and the output signal is detected by second electrodes in the MEA contacting the neuronal cell culture at a second location.

9. The computing system of claim 7, wherein the input signal and the output signal are provided and detected by one or more bidirectional electrodes at the same or different locations.

10. The computing system of claim 1, wherein the computing system is configured such that the neuronal cell culture functions as a reservoir in reservoir computing.

11. The computing system of claim 10, wherein the electronic computing device implements a trainable learning layer that receives an output signal from the neuronal cell culture.

12. The computing system of claim 11, wherein the neuronal cell culture and the trainable learning layer together are configured to function as a recurrent neural network (RNN).

13. The computing system of claim 10, wherein the reservoir is configured to map input signals received from the input device into a different computational space.

14. The computing system of claim 1, wherein the computing system is configured such that the neuronal cell culture functions as a spiking neural network (SNN).

15. The computing system of claim 14, wherein activation potentials of neurons in the neuronal cell culture provides signals for the SNN.

16. A method of using a neuronal cell culture as a compute substrate comprising:
providing, by circuitry coupled to an electronic computing device, an input signal to the neuronal cell culture;
receiving, by the circuitry coupled to the electronic computing device, an output signal from the neuronal cell culture; and
performing a computational task by the electronic computing device based on a difference between the input signal to the neuronal cell culture and the output signal from the neuronal cell culture.

17. The method of claim 16, wherein the input signal is provided by a first electrode in contact with the neuronal cell culture at a first location and the output signal is received by a second electrode in contact with the neuronal cell culture at a second location or the input signal and output signal are provided and received by one or more bidirectional electrodes.

18. The method of claim 16, wherein information in the input signal is encoded by spatial encoding.

19. The method of claim 16, wherein information in the input signal is encoded by temporal encoding.

20. The method of claim 16, wherein in performing the computational task the neuronal cell culture functions as a RNN or SNN.
